(19) United States
(12) Patent Application Publication
(10) Pub. No.: US 2006/0002135 A1
(43) Pub. Date: Jan. 5, 2006

(54) Dental lamp particularly for medical and dental surgeries

(75) Inventors: Alessandro Kokeny, Monterenzio (IT); Domenico Brunetti, Bologna (IT)

Correspondence Address: MODIANO & ASSOCIATI, Via Meravigli, 16, MILAN, 20123, IT

(73) Assignee: EURODENT S.P.A.

(21) Appl. No.: 11/148,241

(22) Filed: Jun. 9, 2005

(30) Foreign Application Priority Data
Jul. 1, 2004 (IT) BO2004A000412

(51) Int. Cl. A61B 1/06

(52) U.S. Cl. 362/573

(57) ABSTRACT

A dental lamp particularly for medical and dental surgeries, comprising an orientable arm that is rigidly coupled at one of its ends to a fixed structure and has the opposite end rotatably associated with a box-like body provided with a screen and a light source. The light source comprises a plurality of luminescent diodes, known as LEDs, which are aligned with respective guides for the emitted light beam, the beam emission directions of all the diodes being substantially convergent.

The present invention relates to a dental lamp particularly for medical and dental surgeries.

### BACKGROUND OF THE INVENTION

In dental surgeries it is necessary to supplement the diffuse ambient light with a beam of light that is localized on the part of the patient to be analyzed (in this case, the mouth).

The light source required to provide this type of localized light is therefore a orientable lamp, which is generally mounted on a movable arm (so as to be able to direct the light beam exactly in the chosen point) with an intensity, frequency and color temperature of the emitted beam that are clearly defined by standard parameters.

Currently applicable standards prescribe conditions regarding the light spot generated by the lamp on a surface that is at a distance of 700 mm (reference plane) from the emission point. The luminous intensity must be comprised between 75% and 100% of the maximum luminous intensity within a circle having a diameter of 50 mm that is centered in the center of the spot (which is elliptical). In the region comprised between said circle and the perimeter of the elliptical spot, the intensity must be comprised between 50% and 75% of the maximum value. At a distance of 60 mm from the center of the elliptical spot, the intensity must be lower than 1200 lux.

The light beam emitted by a halogen lamp can be adjusted by varying the voltage or by adjusting the position of the reflective screen.

The light beam that reaches said spot from the lamp body must be such that the shadow produced by a metallic disk having a diameter of 20 mm and a thickness of 1 mm placed at 50 mm from the reference plane is a dark spot with a maximum diameter of 12 mm.

The frequency and the color temperature are closely mutually correlated parameters, since a warm light, which corresponds to lower frequency values (with a lower limit constituted by the infrared), is associated with lower temperatures than a cold light, which corresponds to higher frequency values (with the ultraviolet as an upper limit).

The dental surgeon must recognize unequivocally the color of a tooth for which a prosthesis is to be prepared (or which he must reconstruct in some way); the light with which the mouth is lit significantly alters color perception, and therefore the standards require the light to be white with a color temperature that the standards specify as comprised between 3600 K and 6500 K (with a preferable intermediate band comprised between 5000 and 5500 K).

Usually, the light sources used inside lamps for the dental sector of this type are halogen lamps, which are able to provide good beam intensity and, if appropriately directed by a suitable parabolic reflector, allow to obtain a highly concentrated illuminated portion (according to the requirements of the standards).

It should be noted that the color temperature of the light beam emitted by a halogen lamp generally depends in a strict sense on the hours of operation and on the type of treatment performed on the parabolic reflector used to direct said beam.

Halogen lamps generate a large amount of heat in all directions: part of this heat entails rapid overheating of the lamp body, and part of the remaining heat is emitted in the direction of the light beam.

Overheating of the lamp body is extremely negative for correct operation, since the components age precociously due to the thermal stress and further work in conditions that are far from the ones that provide maximum efficiency. In order to obviate this drawback, a ventilation element is usually installed inside the lamp body in order to remove the maximum possible amount of heat and convey it outside said body. The presence of this element entails increases in size (due to the installation spaces but also to the provision of the air circuit), from the point of view of energy consumption (caused by the electric power consumption of the motor that drives the fan), in weight (due to the insertion of all the components of the cooling circuit) and most of all noise.

The heat emitted in the direction of the patient, in addition to being unpleasant for the patient (who is, however at a distance by virtue of which he/she is affected only by a limited fraction of the amount of heat released), is negative for the surgeon, who by having the lamp at a short distance from the back of his head is subjected to the maximum intensity of emitted heat.

Moreover, the light emitted by halogen lamps also comprises various components that belong to the ultraviolet frequency: the materials currently used (for example in fillings) are polymeric materials that cure easily if they are subjected to light radiation of a certain frequency, particularly the ultraviolet.

Therefore, when the surgeon operates by using these materials, he cannot use the orientable lamp with complete ease, since said lamp reduces considerably the curing times of said materials.

The average life of halogen lamps is rather short, and accordingly said lamps must be replaced frequently; further, in the final step of their life their light flux degrades considerably and sometimes does not fall within the parameters set by the standards.

### SUMMARY OF THE INVENTION

The aim of the present invention is to obviate the cited drawbacks and meet the mentioned requirements, by providing a dental lamp particularly for medical and dental surgeries that complies with currently applicable statutory provisions and has a low heat emission.

Advantageously, an object of the present invention is to have an emitted light of constant intensity during operating hours and during the life of the lamp.

Another object of the present invention is to have an emitted light beam of adjustable intensity.

Another object of the present invention is to have an emitted light beam that is substantially free from infrared components and optionally has a reduced ultraviolet component, in order to avoid the curing of photopolymerizing materials.

Another object of the present invention is to reduce considerably the weights with respect to a conventional lamp by not requiring the presence of parabolic reflectors.

Within this aim and these objects, another object of the present invention is to provide a dental lamp that is simple, relatively easy to provide in practice, safe in use, effective in operation, and has a relatively low cost.

This aim and these and other objects that will become better apparent hereinafter are achieved by the present dental lamp particularly for medical and dental surgeries, of the type that comprises an orientable arm that is rigidly coupled at one of its ends to a fixed structure and has the opposite end rotatably associated with a box-like body provided with a screen and a light source, characterized in that said light source comprises a plurality of luminescent diodes, known as LEDs, which are aligned with respective guides for the emitted light beam, the beam emission directions of all the diodes being substantially convergent.

### BRIEF DESCRIPTION OF THE DRAWINGS

Further characteristics and advantages of the invention will become better apparent from the following detailed description of a preferred but not exclusive embodiment of a dental lamp particularly for medical and dental surgeries, illustrated by way of non-limiting example in the accompanying drawings, wherein:

FIG. 1 is a schematic perspective view of an apparatus for dental examinations, on which a lamp according to the invention is mounted;

FIG. 2 is a plan view of a lamp according to the invention;

FIG. 3 is a sectional view, taken along the line III-III of FIG. 2, of a lamp according to the invention;

FIG. 4 is a sectional view, taken along the line IV-IV of FIG. 2, of a lamp according to the invention;

FIG. 5 is an enlarged-scale view of the detail V of FIG. 3.

### DESCRIPTION OF THE PREFERRED EMBODIMENTS

With reference to the figures, the reference numeral 1 generally designates a dental lamp particularly for medical and dental surgeries.

The dental lamp 1 is suitable for installation on apparatuses of dental studies that are constituted by a footing 2, which rests on the ground and through which they are connected to the electrical, hydraulic and compressed air supplies, by a chair 3 (mounted on the footing 2), and by a plurality of auxiliary elements for the surgeon.

The footing 2 is generally suitable to move vertically the components that are associated with it by way of the presence of traction elements (not shown in the figure), which are generally constituted by an electric motor and corresponding gears or by a hydraulic piston.

The chair 3 on which the patient lies comprises a seat 4, a back 5 and a headrest 6, all of which can move with respect to each other in order to meet the requirements of the patients and of the surgeon.

Laterally to the chair 3 and rigidly with respect to the footing 2 there is a column 7, in which there is a management and control unit, which is not shown in the figure.

At the top of the column 7 there is usually a basin 8; respective supporting arms 9 and 10 extend from the lateral surfaces thereof, and the first arm 9 is associated with an instrument supporting assembly 11 and the second arm 10 is associated with an aspirator assembly 12.

At the top of the column 7, proximate to the basin 8, an arm 13 is rotatably coupled, is provided with articulations 14 in order to facilitate its movement, and rotatably supports the lamp 1 at its free end.

The dental lamp 1 is comprised within a box-like body 15, which is provided with a screen 16; the box-like body 15 internally accommodates a light source comprising a plurality of luminescent diodes 17, known as LEDs, in which the respective beam emission directions substantially converge.

The screen 16 is designed to protect all the components installed inside the box-like body 15 against any unwanted impacts or contacts: in the described embodiment, said screen has an elliptical base and is made of a material such as plastics with a thickness comprised between 2 and 5 mm. The screen 16 is fixed on the box-like body 15 by means of removable retention elements, which are not shown in the figure.

The diodes 17 of the type used emit a white light beam with a color temperature of approximately 5500 K, are voltage- and current-adjustable, and have an average life on the order of 50,000 hours.

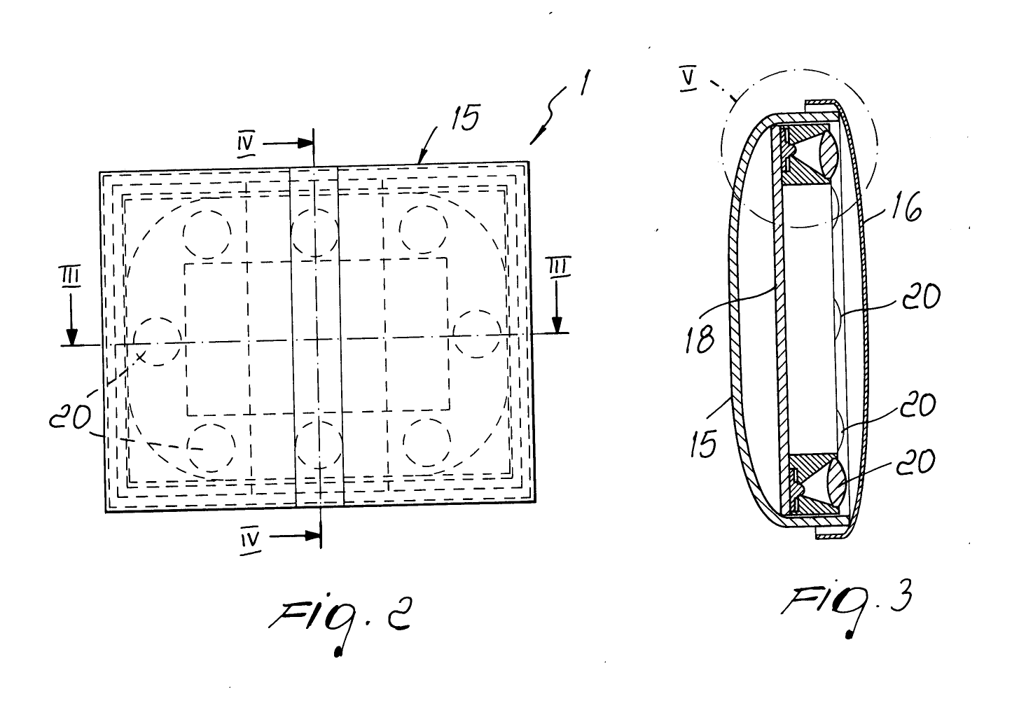

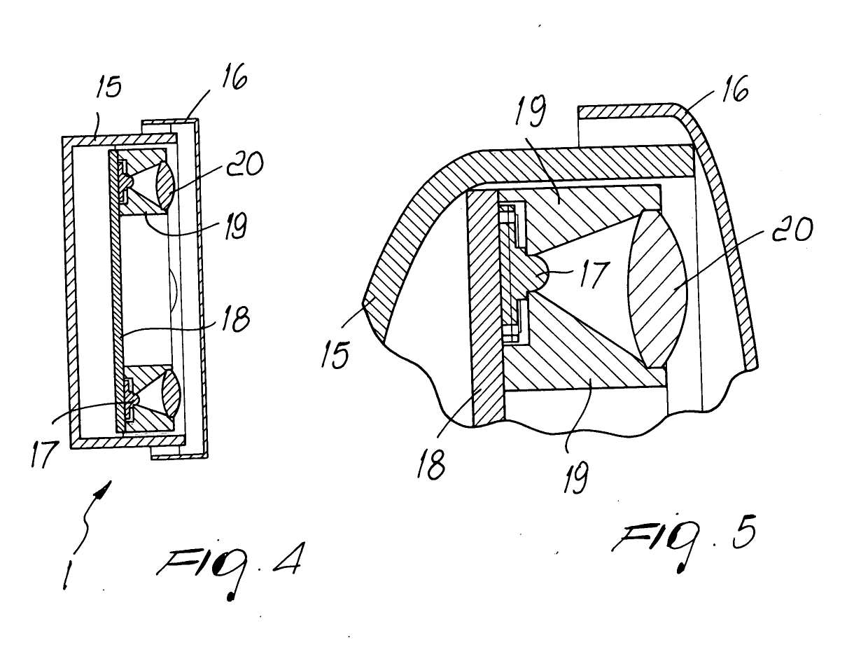

In the embodiment described in FIG. 2, there are eight diodes 17, arranged substantially along the perimeter of an ellipse. Optionally, other diodes 17 might be added to the configuration shown in FIG. 2. Each one of the light-emitting diodes 17 used for the embodiment described has a power of three watts: the choice of this power is due to a compromise between good intensity of the emitted light beam and low heating of said diodes during operation.

As already mentioned, the light-emitting diodes 17 emit a beam of light with a color temperature of substantially 5500 K, a value which is within the range prescribed by the standards; this color temperature ensures that the emitted light is almost perfectly white, ensuring that the operator has optimum conditions for distinguishing colors.

This color temperature ensures that the light-emitting diodes 17 emit a light beam with a frequency that is substantially lower than the ultraviolet and substantially higher than the infrared: this reduces the risk of facilitating the curing of the photopolymerizing polymers (which are sensitive to the ultraviolet) used for certain dental treatments and also avoids biasing the perception of colors and reduces the heating of the face of the patient due to radiation.

The diodes 17 are mounted on a common base 18, in which the light-emitting end is arranged within a light guide such as a light guiding cone 19, and are aligned with respective lenses 20, which are fixed frontally to the screen 16 in the box-like body 15. The base 18 is a common board for wiring electronic circuits, and on said board it is convenient in certain cases to arrange finned surfaces in order to dissipate the heat generated by the power supply components. The power supply values (voltage and current) must allow control and modulation in order to allow to vary the intensity of the light beam (by varying the intensity of the current), and the components for conditioning the power supply are fundamental, since the diodes 17 require stabilized input values.

The light-emitting diodes 17 are connected electrically in series, and a Zener diode is connected in parallel on each one and is intended to allow electric current to flow if the light emitting diode fails: it is thus possible to ensure the operation of the lamp, albeit in a reduced mode, if some light-emitting diodes 17 are not working.

The lenses 20 are fixed to the light guiding cone 19 and are optionally inclined with respect to it, and therefore are offset axially with respect to the corresponding diodes 17.

The light guiding cones 19 may be provided as a single ring (which comprises them all), generally made of a material such as plastics, with a substantially elliptical base; the exemplifying thickness of said ring is lower than 25 mm and the lower face is arranged above the base 18, with the holes at the diodes 17 (holes provided with a diameter that approximates the diameter of the diodes 17) mounted on the base 18. The upper face of the ring has holes that are suitable to accommodate the lenses 20. The axis of each one of the elementary cones 19 is inclined with respect to the axis of the ring, and said inclination entails that the lenses 20 are axially offset with respect to the diodes 17 by a length comprised between 1 and 10 mm; further, the seats of the lenses 20 may accommodate the lenses 20 so that they are arranged at an angle comprised between 0° and 10°.

Once the lenses 20 have been arranged in the respective seats of the ring, said lenses must be locked by way of appropriate gaskets (or other equivalent fixing means); the lenses 20 of the type used in the particular embodiment described in the figure are biconvex and have a diameter comprised between 20 and 24 mm.

It may be convenient to insert a ventilation element inside the box-like body 15 in order to remove the maximum possible amount of heat, so as to make all the components work in optimum temperature conditions.

The operation of the invention is intuitive: by orienting the lamp 1 toward the patient, the light beam must strike the patient so as to illuminate with maximum intensity the mouth, while having a reduced emission intensity at the eyes of the patient (as required by the standards).

The arrangement of the diodes 17 ensures that the lighting of the mouth of the patient has an extremely uniform distribution, thus obtaining a lamp 1 whose characteristics exceed by far the requirements of the standards. Further, the lamp 1, as a consequence of the minimal amount of heat emitted by the diodes 17, might also be provided without electromechanical heat dissipation systems, thus reducing manufacturing costs and overall weight.

The screen 16, by not having to provide a filtering action on the emitted light beam, can also be provided with a reduced thickness and with transparent materials having a low relative density, with the advantage of further reducing the weight of the lamp 1.

It has thus been shown that the invention achieves the intended aim and objects.

The invention thus conceived is susceptible of numerous modifications and variations, all of which are within the scope of the appended claims.

For example, the number of diodes 17 used for each lamp 1 can also be lower than the described number (eight or more diodes 17).

By using diodes 17 that have a different (higher) power than the selected ones it is possible to achieve the same light beam intensity: the concentration of the beam in the area prescribed by the standards in this case is obtained by appropriately shaping the guiding cones 19 or by providing a common parabolic reflector that concentrates the light projection.

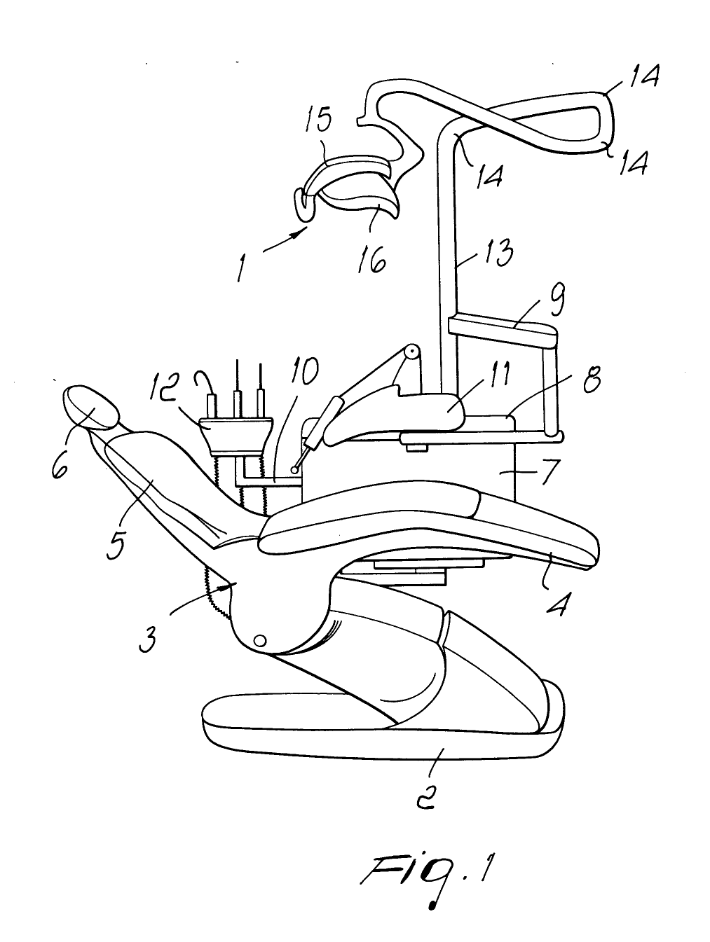

The lamp 1 can also be mounted on the wall, floor or ceiling (it is therefore not necessary to install it in dental surgery apparatuses such as the one shown in FIG. 1).

All the details may further be replaced with other technically equivalent ones.

In the described embodiments, individual characteristics, given in relation to specific examples, may actually be interchanged with other different characteristics that exist in other embodiments.

Moreover, it is noted that anything found to be already known during the patenting process is understood not to be claimed and to be the subject of a disclaimer.

The embodiment of the present invention shall be carried out in the most scrupulous compliance with the statutory and regulatory provisions related to the products of the invention or correlated thereto and following any required authorization of the corresponding competent authorities, with particular reference to regulations related to safety, environmental pollution and health.

In practice, the materials used, as well as the shapes and the dimensions, maybe any according to requirements without thereby abandoning the scope of the protection of the appended claims.

The disclosures in Italian Patent Application No. B02004A000412 from which this application claims priority are incorporated herein by reference.

What is claimed is:

1. A dental lamp for medical and dental surgeries, that comprises: a fixed structure; a box-shaped body provided with a screen and with a light source; an orientable arm having first and second opposite ends, with the first end rigidly coupled at said fixed structure and the second, opposite end rotatably associated with said box-shaped body; and light guides for the emitted light beam, said light source comprising a plurality of luminescent diodes, known as LEDs, aligned with respective ones of said light guides and wherein beam emission directions of all said diodes are substantially convergent.

2. The lamp of claim 1, comprising at least three of said light-emitting diodes arranged to form corners of a polygon.

3. The lamp of claim 2, comprising an additional light emitting diode arranged inside said polygon.

4. The lamp of claim 1, comprising at least five of said light-emitting diodes arranged so as to form a perimeter of an ellipse.

5. The lamp of claim 4, comprising additional light-emitting diodes that are partially arranged inside said ellipse.

6. The lamp of claim 4, comprising eight of said light-emitting diodes.

7. The lamp of claim 1, comprising lenses which are fixed frontally to said screen in said box-shaped body, said plurality of diodes being mounted on a common base and being aligned with respective ones of said lenses.

8. The lamp of claim 7, wherein said lenses are fixed so as to be axially offset and arrangeable inclined with respect to said guide for directing adequately the beam emitted by a respective one of said plurality of diodes.

9. The lamp of claim 1, wherein each one of said plurality of light-emitting diodes has a power of three watts.

10. The lamp of claim 1, wherein each one of said plurality of light-emitting diodes is adapted to emit a beam of light with a color temperature of substantially 5500 K.

11. The lamp of claim 1, wherein each one of said plurality of light-emitting diodes is adapted to emit a light beam with a frequency that is substantially lower than a ultraviolet frequency and substantially higher than an infrared frequency.

12. The lamp of claim 1, wherein said box-shaped body comprises a heat dissipation element.
